(19) United States
(12) Patent Application Publication
(10) Pub. No.: US 2011/0060135 A1
(43) Pub. Date: Mar. 10, 2011

(54) Selective Purification of Small RNAs from Mixtures

(75) Inventors: George Tzertzinis, Cambridge, MA (US); Jean-Etienne Morlighem, Yokohama (JP)

(73) Assignee: NEW ENGLAND BIOLABS, INC., Ipswich, MA (US)

(21) Appl. No.: 12/744,938

(22) PCT Filed: Nov. 18, 2008
(86) PCT No.: PCT/US08/83846

§ 371 Date: Nov. 22, 2010

Related U.S. Application Data
(60) Provisional application No. 60/991,083, filed on Nov. 29, 2007.

(51) Int. Cl. C07H 21/02; B01J 19/12

(52) U.S. Cl. 536/23.1; 422/527

(57) ABSTRACT

Methods and kits are provided for obtaining small RNAs from a mixture of RNAs of varying sizes such as can be found in a cell lysate or an enzyme-digested RNA. The methods and kits utilize magnetic beads and require the addition of one or more alcohols to bind small RNAs effectively to the beads.

### BACKGROUND

RNA purification methods are optimized for purification of high molecular weight polynucleotides, and result in low recovery of low molecular weight polynucleotides. There is a growing need for the isolation and purification of low molecular weight polynucleotides, such as small RNA, for example, microRNA and fragmented DNA, for a range of uses in molecular biology research and in the study of disease processes in cells.

Methods for non-specific binding of nucleic acid to magnetic particles induced by precipitation using polyethylene glycol (PEG) and salt have been described in U.S. Pat. Nos. 6,534,262 and 5,705,628 and by Hawkins, et. al. (Nucleic Acids Res. 23:4742-4743 (1995). However, magnetic particle-based technologies have also been used more generally for automated separation of analytes (see for example U.S. Pat. No. 4,935,147 and DNA Sequencing II: Optimizing Preparation and Cleanup, ed. Kieleczawa, Ch. 9, pub. Jones and Bartlett, Sudbury, Mass., 2006).

The major drawback of the various methods developed thus far is their inefficiency with respect to the purification and recovery of RNAs having a size less than 50 nucleotides.

### SUMMARY

In an embodiment of the invention, a method is provided that includes the steps of (a) combining in a reaction vessel, a set of magnetic beads, such as carboxylated magnetic beads, and a solution containing PEG, a salt and a plurality of RNA molecules of various sizes, for binding large RNA molecules to the set of magnetic beads; (b) separating the RNA molecules in the solution from the RNA molecules bound to the set of magnetic beads using for example an external magnet and optionally repeating step (a) to ensure binding of as much large RNA as possible from the mixture; (c) adding an additional set of magnetic beads together with one or more alcohols such as ethanol and/or isopropanol for binding to the RNA molecules; and (d) separating the magnetic beads from the solution. The RNA molecules may be eluted using an aqueous solution containing less than 0.2M salt added to the isolated beads.

The plurality of RNA molecules may be in a cell lysate or derived therefrom or may result from RNase cleavage of large dsRNA or any in vivo or in vitro source of RNA. The plurality of RNA molecules of various sizes may consist of single-stranded RNA (ssRNA), double-stranded RNA (dsRNA) or a mixture of the two.

A further embodiment of the method includes (a) mixing a cell lysate containing RNA with a purification reagent containing magnetic beads, PEG, salt and one or more alcohols; (b) allowing the RNA to bind to the magnetic beads; and (c) applying an external magnet to the beads for separating the RNA from the lysate.

In a further embodiment, a method is provided that includes the steps of (a) mixing a cell lysate with a purification reagent containing PEG, a salt and a first set of magnetic beads, such that the RNA molecules greater than 50 nucleotides are bound to the first set of beads; (b) applying an external magnet to the first set of beads for separating the large RNA from the lysate; and (c) permitting the unbound RNA in the lysate to bind to a second set of magnetic beads by adding one or more alcohols. This enables RNA molecules having a size of less than 50 nucleotides to bind to the magnetic beads. The RNA can then be eluted from the RNA from the second set of beads.

A further embodiment of the invention provides a kit containing magnetic beads, a reaction vessel, a solution containing PEG, a salt, a wash solution, an elution solution, instructions describing the method above and optionally a magnet.

### BRIEF DESCRIPTION OF THE DRAWINGS

FIG. 1 shows a flowchart which includes two solid phase binding steps for isolating small RNA molecules from a crude mixture.

Step 1 shows mixing of a crude sample such as a lysate containing RNA (dsRNA or ssRNA or a mixture of both) of varying sizes with a first set of magnetic beads and a solution containing PEG and a salt to bind the large RNAs.

Step 2 shows binding of large RNAs to the first set of magnetic beads while the unbound RNAs remain in the supernatant.

Step 3 shows binding of small RNA to an additional set of magnetic beads by mixing of the supernatant from step 2 with magnetic beads in the presence of one or more alcohol solutions.

Step 4 shows attraction of the magnetic beads in the reaction vessel to an external magnet to permit isolation of small RNA from unbound material.

Step 5 shows elution of the small RNAs from the beads after addition of water or a low salt solution.

FIG. 2 shows how binding of small dsRNA (below 50 bp) requires a two-step protocol. Lane 1 shows a mixture of dsRNA fragments with sizes between 21 and 500 as the starting material. Binding was done in the presence of 20% PEG 6000 (lanes 2 and 3), 8000 (lanes 4 and 5) and 10000 (lanes 6 and 7) and 1.25M NaCl. The material was eluted with water from beads after binding to a first set of beads (lanes 2, 4, 6) or the unbound material was applied to a second set of beads in the presence of 60% ethanol before elution (lanes 3, 5, 7).

FIG. 3 shows the conditions required to capture small dsRNA on beads using different combinations of 20% PEG and alcohols.

Lane 1 contains small dsRNA (18-22 bp) before adding to beads.
Lane 2 contains the small dsRNA eluted from beads after binding in PEG 6000/ethanol.
Lane 3 contains the small dsRNA eluted from beads after binding in PEG 8000/ethanol.
Lane 4 contains the small dsRNA eluted from beads after binding in PEG 10000/ethanol.
Lane 5 contains the small dsRNA eluted from beads after binding in PEG 12000/ethanol.
Lane 6 contains the small dsRNA eluted from beads after binding in PEG 6000/isopropanol.
Lane 7 contains the small dsRNA eluted from beads after binding in PEG 8000/isopropanol.
Lane 8 contains the small dsRNA eluted from beads after binding in PEG 10000/isopropanol.
Lane 9 contains the small dsRNA eluted from beads after binding in PEG 12000/isopropanol.

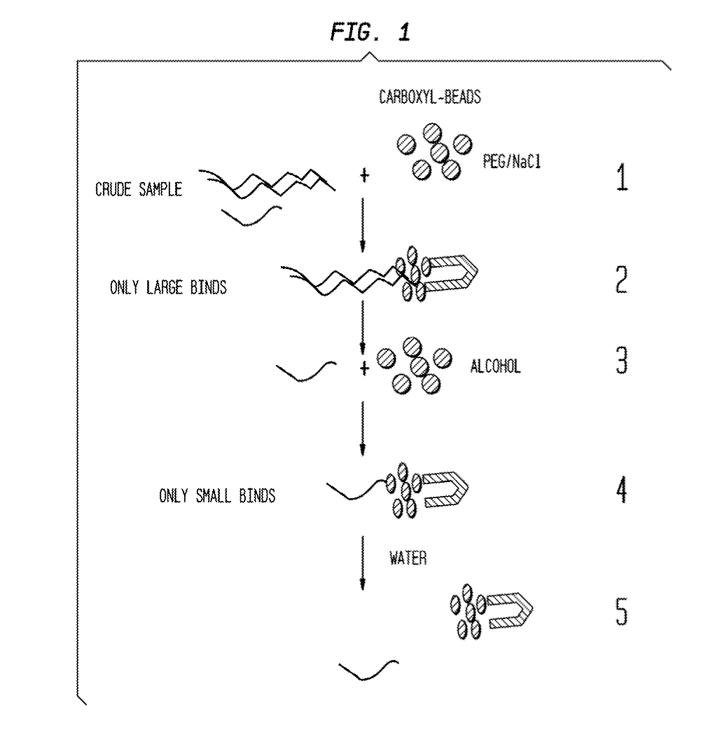

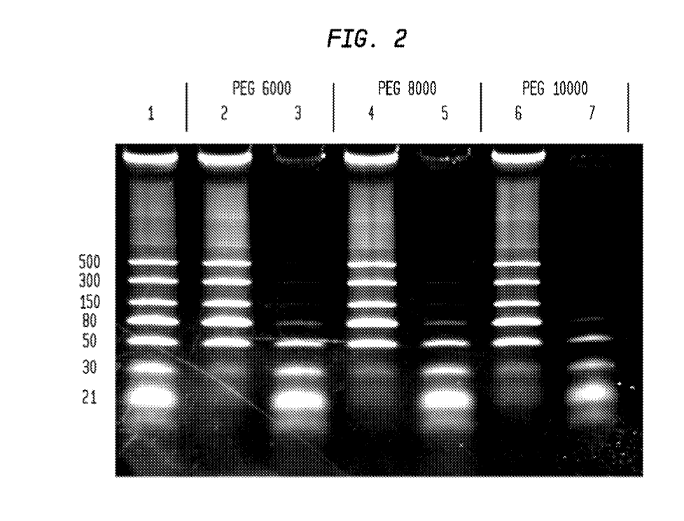

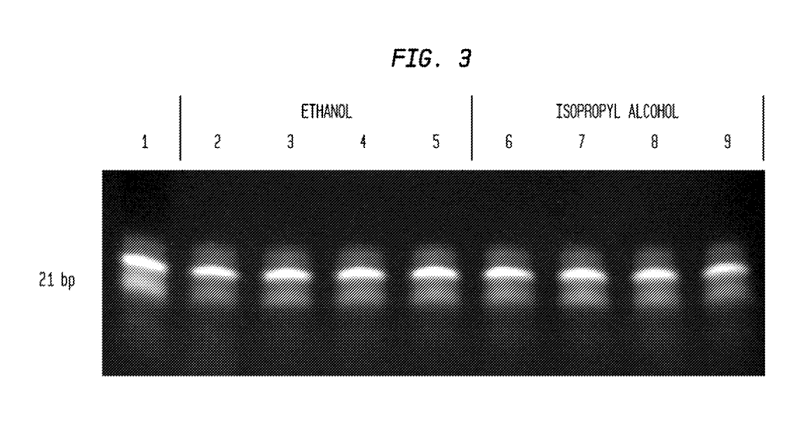

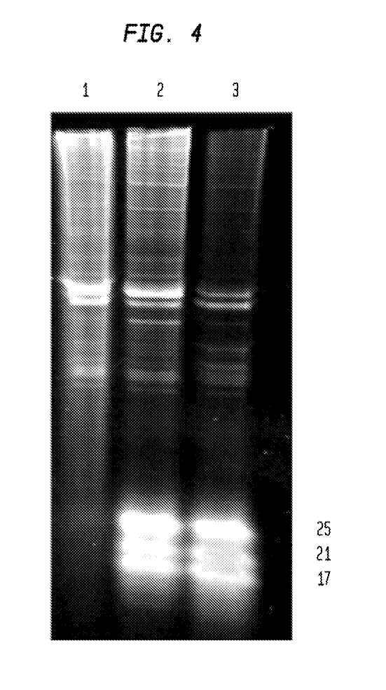

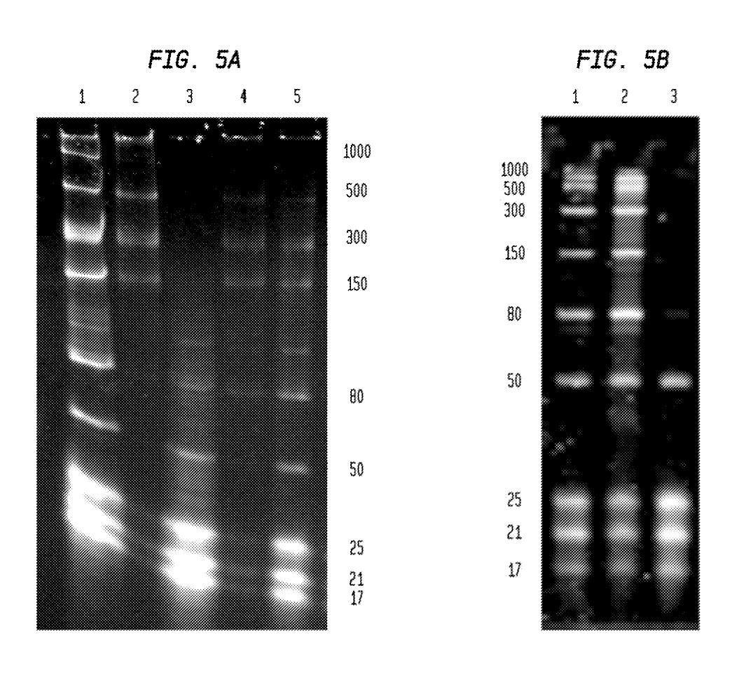

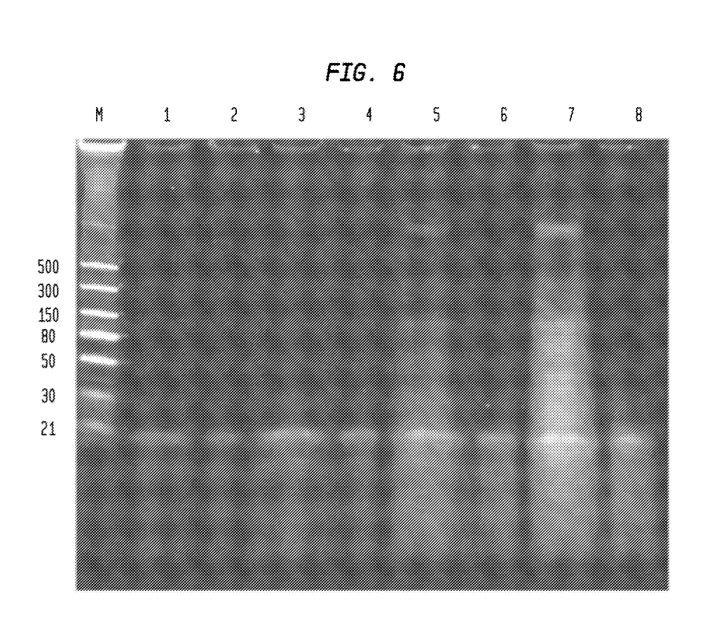

FIG. 4 shows binding of short ssRNA to the beads from a crude total RNA sample using the protocol described in FIG. 1. The polyacrylamide gel was stained with SYBR Gold.

Lane 1 contains RNA eluted from a first set of magnetic beads (20% PEG and 1.25M NaCl).
Lane 2 contains input material containing total RNA from HeLa cells spiked with three ssRNA of 17, 21 and 25 nucleotides in length.
Lane 3 contains RNA eluted in distilled water from a second set of magnetic beads after binding of the RNA to the beads in the presence of 20% PEG and ethanol.

FIGS. 5A and 5B show improved size-separation of small ssRNAs from large ssRNAs in a multi-step PEG/NaCl protocol using magnetic beads.

FIG. 5A shows the results using a native 20% polyacrylamide gel stained with SYBR Gold.

Lane 1 is a marker with ssRNA of 17, 21, 25, 50, 80, 150, 300, 500 and 1000 nucleotides in length.
Lane 2 contains RNA after elution in distilled water from a first set of magnetic beads to which the RNA was bound using 10% PEG 6000, 1.25 M NaCl.
Lane 3 contains RNA bound to a second set of magnetic beads in the presence of 20% PEG 6000 and ethanol and then eluted with water.
Lane 4 contains RNA after elution from a first set of magnetic beads after binding of RNA to beads in the presence of 20% PEG 6000, 1.25 M NaCl.
Lane 5 contains RNA first bound to a second set of magnetic beads in the presence of 20% PEG 6000 and ethanol and then eluted with water.

FIG. 5B shows the results of an analysis using a denaturing Urea/15% polyacrylamide gel stained with SYBR Gold.

Lane 1 contains input ssRNA of 17, 21, 25, 50, 80, 150, 300, 500 and 1000 nucleotides in length.
Lane 2 contains RNA bound in the first step (20% PEG, 1.25 M NaCl).
Lane 3 contains RNA eluted from the second set beads after binding in the presence of 20% PEG, and ethanol.

FIG. 6 shows isolation of siRNA from crude RNAseIII digestions of long dsRNA performed under different enzyme to substrate ratios. The reaction product size range can be optimized by adjusting the enzyme to substrate ratio (RNaseIII/dsRNA), and manganese ion concentration (see for example U.S. Publication No. 2004-0038278). Lanes 1, 3, 5 and 7 show crude reaction mixture before purification. Lanes 2, 4, 6 and 8 show the product obtained after following the two-step bead purification method described in the Examples. The ratios used here are as follows: Lanes 1 and 2: 1 μl RNAseIII (New England Biolabs, Inc. (NEB, Ipswich)):0.6 μg dsRNA; lanes 3 and 4: 1 μl RNAseIII (NEB, Ipswich):1.2 μg dsRNA; lanes 5 and 6: 1 μl RNAseIII (NEB, Ipswich):1.8 μg dsRNA; and lanes 7 and 8: 1 μl RNAseIII (NEB, Ipswich):2.4 μg dsRNA. Lane M contains a dsRNA size marker.

### DETAILED DESCRIPTION OF THE EMBODIMENTS

An embodiment of the method includes at least one of the following steps:

1. Obtaining magnetic beads which are coated with a functional group;

2. Adding a sample, containing a mixture of sizes of RNAs, to the beads for binding large RNA (greater than 50 nucleotides) in the presence of PEG and salt, wherein the target RNA is small ssRNA or dsRNA of size less than 50 bases;

3. Optionally adding additional coated magnetic beads to the unbound material one or more times as required until the unwanted large material is satisfactorily removed from the target RNA as determined, by for example, gel electrophoresis;

4. Mixing unbound RNA with a fresh preparation of coated magnetic beads in the presence of one or more alcohols so that the target RNA now binds the magnetic beads;

5. Collecting the beads, washing the beads and eluting target RNA from the beads using, for example, water or a low salt aqueous buffer; and

6. Using target RNA for gene silencing, detection analysis, cloning or other uses.

In another embodiment, the total RNA containing large and small fragments of RNA may be separated from a mixture of molecules such as a cell lysate in one binding step. This step involves adding magnetic beads, PEG, salt and one or more alcohols to the mixture of molecules to efficiently bind both large and small RNAs.

“Small RNA” is intended to mean RNA having a size of approximately 50 nucleotides or less.

“Large RNA” is generally intended to mean RNA having a size greater than 50 nucleotides.

“Low salt concentration” is intended to mean a salt concentration that is generally less than about 200 mM.

Large and small RNA can be readily eluted from magnetic beads using an aqueous solution containing low salt or in water only.

Magnetic beads may be carboxylated. They may also be derivatized with amine or another charged group.

Salts used in the binding solution can include any of NaCl, KCl, LiCl, sodium acetate, sodium dodecyl sulfate, lithium dodecyl sulfate, potassium acetate, guanidinium chloride, guanidinium isothiocyanate. An example of a salt solution suitable for binding RNA is 1.25M NaCl or 500 mM LiCl, 0.5% sodium dodecyl sulfate.

PEG or another short chain polymeric alcohol can be used for binding RNA to beads, including any of PEG 2000, PEG 4000, PEG 6000, PEG 8000, PEG 10000, PEG 12000, PEG 14000, PEG 20000 or PEG 35000. Lower molecular weight polymers are preferred for their lower viscosity properties.

The one or more alcohols for the second binding step include ethanol, propanol, and/or isopropanol. The alcohol may be used in an amount of less than 70% final concentration, such as 60% final concentration or less.

Elution solutions include water or any low ionic strength solution compatible with downstream uses of the material such as TE (20 mM Tris-HCl, 1 mM EDTA).

While large reaction vessels may be used, all the binding wash and elution steps can be performed in small reaction tubes such as eppendorf tubes, or in microtiter plates of different sizes as long as the appropriate application of a magnet or electromagnet permits separation of the magnetic beads.

Embodiments of the method allow enrichment of biological samples for small RNAs which include, siRNA, microRNA, piRNA, rasiRNA or other unidentified small RNA for characterization. The crude sample can be a cell lysate or biological fluid, or an enzymatic reaction mixture that contains small RNA. The use of magnetic particles allows scalability and compatibility with high throughput applications. The purified product eluted in low salt solution is compatible in downstream applications which include detection by hybridization or RTPCR/QPCR methods, labeling for microarray analysis, expression profiling, ligation, sequencing, etc. Other uses include the purification of small RNA enzymatic products such as siRNAs after Dicer or RNAseIII processing reactions in microtiter plate format for example in large scale RNAi screen applications (Kittler et al. Nature 432:1036-1040 (2004)).

All references cited above and below, as well as U.S. provisional application No. 60/991,083 filed Nov. 29, 2007, are herein incorporated by reference.

### EXAMPLES

### Example 1

### Purification of Small dsRNAs

The following procedure was used to perform the experiments. A sample size of 50 μg of RNA was used.

### Preparation of Beads

150-250 μg of Sera-Mag MG-CM Seradyn beads (50 mg/mL) were aliquoted into a clean RNase-free tube. 100 μL of solution A (0.5M EDTA, pH 8.0) was added. Beads were exposed to a magnet (S1506S NEB, Ipswich, Mass.). The wash was repeated (Steps 2 & 3 in FIG. 1). The beads were re-suspended in 9 μl of Solution B (2.5M NaCl, 0.5M EDTA, pH 8.0) and kept at 4° C. for storage.

### Purification of RNA

3 μl of washed beads (150 μg) were aliquoted into a clean RNase-free tube. 20-50 μl of RNA sample (up to 50 μg) were added to the beads. 1 sample-volume of Solution C(40% PEG 6000, 2.5 M NaCl) was added to the beads followed by an incubation at room temperature for 5 minutes. The sample was then exposed to a magnet for bead separation and the unbound material in the supernatant transferred to a clean RNase-free tube. 3 μl of washed beads (150 μg) was added to the recovered supernatant followed by an incubation at room temperature for 5 minutes. Again the sample was exposed to a magnet and the unbound material in the supernatant transferred into a new clean RNase-free tube.

3 μl of washed beads (150 μg) was added to the recovered supernatant, followed by 3 sample volumes of 100% ethanol and incubated at room temperature for 10 minutes. The preparation was then placed on a magnet and the supernatant discarded. 150 μl of Solution D (70% ethanol) was added to the beads, the sample exposed to a magnet and the solution was discarded. This time, the magnetic beads bound to RNA were collected, air-dried at room temperature for 5 minutes, re-suspended in 30 μl distilled water, incubated at room temperature for 5 minutes, exposed to a magnet, and the eluted material containing the small RNA collected.

### SOLUTIONS:

### Solution A: 0.5M EDTA, pH 8.0

### Solution B: 2.5 M NaCl, 0.5 MEDTA pH 8.0

### Solution C: 40% PEG-6000, 2.5 M NaCl

Solution D: 70% ethanol in water

### Example 2

### Purification of siRNA from a Mixture

The procedure was performed as above using 0.5 μg of the dsRNA marker (N0363, NEB, Ipswich, Mass.) supplemented with a siRNA mixture (N2006, NEB, Ipswich, Mass.) formed by digestion of a dsRNA with RNaseIII. In addition, Solution C contained 40% of either PEG 6000, PEG 8000 or PEG 10000; Only two sets of beads were used. The material was recovered from the first set of beads (large RNA). The material was also recovered from the second set of beads to which ethanol was added (small RNA). The RNA obtained from the different sets of beads was analyzed in 20% polyacrylamide gels. The results are shown in FIG. 2.

### Example 3

### Determining Efficiency of Recovery of Small RNA Using Different Sizes of PEG

30 μL (300 μg) of prepared beads were mixed with 150 ng of siRNA mixture (N2006, NEB, Ipswich, Mass.) in 20% PEG 6000, 8000, 10000 or 12000 and 50% ethanol or isopropanol final concentration. The results are shown in FIG. 3. Quantification of RNA was obtained by comparing the purified product with different amounts of the siRNA Marker (N2101S, NEB, Ipswich, Mass.) on a 20% TBE Gel (Invitrogen, Carlsbad, Calif.) stained with SYBR Gold. The quantification was performed using ImageQuantTL® software after scanning of the gel on a Typhoon 9400 imager (GE Healthcare, Piscataway, N.J.). The recovered amount yield of siRNA was 80-90% using ethanol in the second step or 65-86% siRNA for isopropanol.

### Example 4

### Determining Efficiency of Recovery of Small RNAs of Varying Sizes from Total RNA

5 μg total RNA was mixed with 2 μg of three ssRNAs of length 17, 21 and 25 nucleotides and used for binding with 150 μg of washed beads using the RNA purification protocol described above. Only two sets of beads were used for large and small RNAs respectively. The RNA was recovered for analysis from the first and second sets of beads. In each case, RNA was mixed with formamide and analyzed in a 20% polyacrylamide gel. The results are shown in FIG. 4.

### Example 5

### Binding of ssRNA to Magnetic Beads

The experiment was done using the two-step protocol of FIG. 1. The input sample was a mixture of ssRNA strands of sizes 17, 21, 25, 50, 80, 150, 300, 500 and 1000 nucleotides. The binding step of the large RNA was performed in the presence of 10% PEG 6000 or 20% PEG 6000. In the former case, 0.6 sample volumes of PEG 6000 was added along with ethanol with the second set of beads. The second set of beads were washed twice with 70% ethanol before elution of the bound small RNA in water. Samples of the elution were analyzed in native 20% polyacrylamide gel or denaturing polyacrylamide/urea gels stained with SYBR Gold. The results are shown in FIG. 5.

### Example 6

### Purification of siRNA from Undigested RNA after RNaseIII Digestion

Various amounts of large RNA was digested with RNAseIII such that at some ratios, the RNA was only partially digested. The purification method described in Example 1 was used to separate the resultant siRNA from partially digested or undigested RNA.

The reaction of RNA with RNaseIII was performed as described in Morlighem et al. Biotechniques 42:599-606 (2007) and processed with the two-step protocol detailed above. The purification of short RNA from all reactions was verified by gel electrophoresis as shown in FIG. 6.

### Example 7

### Enrichment of Small RNAs from Cell Lysates

HeLa cells were used for the purification of small RNAs directly from lysate. Cells grown in 100 mm culture dishes were lysed in 20 mM Tris-HCl pH 7.5, 50 mM LiCl, 0.5% Lithium dodecyl sulphate, 1 mM EDTA, 5 mM DTT. The lysate was applied to carboxylated magnetic beads and the procedure described in Example 1 was followed. The samples were analyzed on polyacrylamide gels. The final obtained material was enriched in small RNAs compared to standard RNA purification methods such as Trizol.

### Example 8

### Purification of Total RNA from a Cell Lysate in a One-Step Purification

A cell lysate is mixed with a preparation of magnetic beads, 20% PEG 6000, 1M NaCl, and 60% ethanol. The beads are washed with 70% ethanol and the RNA of various sizes are eluted in water to provide efficient recovery of the total RNA.

1. A method, comprising:
(a) combining in a reaction vessel, a set of magnetic beads and a solution containing polyethylene glycol (PEG), a salt and a plurality of RNA molecules of various sizes, for binding large RNA molecules to the set of magnetic beads;
(b) separating the RNA molecules in the solution from the RNA molecules bound to the set of magnetic beads and optionally repeating step (a);
(c) adding an additional set of magnetic beads and one or more alcohols for binding to the RNA molecules in the solution; and
(d) separating the magnetic beads from the solution and eluting the RNA molecules therefrom.

2. A method according to claim 1, wherein step (d) further comprises eluting the RNA molecules from the additional set of beads by adding an aqueous solution containing less than 0.2M salt to the beads.

3. A method according to claim 1, wherein the one or more alcohols is ethanol.

4. A method according to claim 1, wherein the magnetic beads are carboxylated beads.

5. A method according to claim 1, wherein separating the beads is achieved by the presence of a magnet on the outside of the reaction vessel.

6. A method according to claim 1, wherein the plurality of RNA of varying sizes is contained in a cell lysate or derived therefrom.

7. A method according to claim 1, wherein the plurality of RNA molecules of varying sizes results from an enzymatic digestion of large RNA.

8. A method according to claim 1, wherein the plurality of RNA molecules is selected from the group consisting of single-stranded RNA (ssRNA), double-stranded RNA (dsRNA) and a mixture of ssRNA and dsRNA.

9. A method, comprising:
(a) mixing a cell lysate containing RNA with a purification reagent containing magnetic beads, PEG, salt and one or more alcohols;
(b) allowing the RNA to bind to the magnetic beads; and
(c) applying an external magnet to the beads for separating the RNA from the lysate.

10. A method, comprising:
(a) mixing a cell lysate with a purification reagent containing PEG, a salt and a first set of magnetic beads, such that the RNA molecules greater than 50 nucleotides are bound to the first set of beads;
(b) applying an external magnet to the first set of beads for separating the beads from the lysate;
(c) permitting unbound RNA in the lysate to bind to a second set of magnetic beads by adding one or more alcohols to the lysate wherein RNA molecules having a size of less than 50 nucleotides are bound to the second set of magnetic beads; and
(d) eluting the RNA from the second set of beads.

11. A method according to claim 10, further comprising eluting the total RNA from the beads by adding water or an aqueous low salt solution.

12. A method according to claim 10, wherein the one or more alcohols is ethanol.

13. A method according to claim 12, wherein the ethanol is present in the purification reagent such that the final concentration is 60% or less.

14. A kit, comprising: magnetic beads, a reaction vessel, a solution containing PEG, a salt, a wash solution, an elution solution, instructions for executing a separation method described in claim 1 or claim 9, and optionally a magnet.
